(19) United States
(12) Patent Application Publication
(10) Pub. No.: US 2024/0325641 A1
(43) Pub. Date: Oct. 3, 2024

(54) A delivery member guard locking member and a sub-assembly of a medicament delivery device

(71) Applicant: SHL Medical AG, Zug (CH)

(72) Inventors: Ming-Ting Yin, Taoyuan City (TW); Philipp Zingel, Taoyuan City (TW); Roman Käslin, Zurich (CH); Lino Manuel Heckhorn Ghilardi, Zurich (CH)

(21) Appl. No.: 18/293,870

(22) PCT Filed: Aug. 3, 2022
(86) PCT No.: PCT/EP2022/071823

§ 371 Date: Jan. 31, 2024

(30) Foreign Application Priority Data
Aug. 9, 2021 (EP) 21190455.2

(51) Int. Cl. A61M 5/20

(52) U.S. Cl. CPC A61M 5/20; A61M 2005/2073

(57) ABSTRACT

A delivery member guard locking member comprising: a body (30; 130) extending in an axial direction along a longitudinal axis (L) between a proximal end and a distal end; wherein the body comprises a plurality of proximally directed surfaces (35, 35′; 135, 135′) extending perpendicular to the longitudinal axis (L) and sequentially arranged next to each other; and wherein each of the plurality of proximally directed surfaces (35, 35′; 135, 135′) is at a different position in the axial direction from the rest of the plurality of proximally directed surfaces (35, 35′; 135, 135′).

### CROSS-REFERENCE TO RELATED APPLICATIONS

The present application is a U.S. National Phase Application pursuant to 35 U.S.C. § 371 of International Application No. PCT/EP2022/071823 filed Aug. 3, 2022, which claims priority to EP patent application Ser. No. 21/190,455.2 filed Aug. 9, 2021. The entire disclosure contents of these applications are herewith incorporated by reference into the present application.

### TECHNICAL FIELD

The present disclosure generally relates to a delivery member guard locking member of a medicament delivery device and to a sub-assembly of a medicament delivery device, and particularly to a delivery member guard locking member with a plurality of proximally directed surfaces and to a sub-assembly comprising a guard adapter and a locking member movable related to the guard adapter.

### BACKGROUND

Medicament delivery devices such as autoinjectors often include a safety sub-assembly for avoiding injury by or contamination of a medicament delivery member or damage of the medicament delivery member before, during and after delivery of a medicament or drug contained in the medicament delivery device.

It has been appreciated that there are solutions for providing safety sub-assemblies with delivery member guards and mechanisms that can lock the delivery member guards after use of the medicament delivery devices. Therefore, a safe operation of the medicament delivery device could be advantageous.

### SUMMARY

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

A first aspect of the present disclosure provides a delivery member guard locking member comprising: a body extending in an axial direction along a longitudinal axis between a proximal end and a distal end; the body comprises a plurality of proximally directed surfaces sequentially arranged next to each other; and each of the plurality of proximally directed surfaces is at a different position in the axial direction from the rest of the plurality of proximally directed surfaces.

Preferably, according to another embodiment, the plurality of proximally directed surfaces extend perpendicularly relative to the longitudinal axis.

Preferably, according to another embodiment, each of the plurality of proximally directed surfaces is offset relative to the rest of the plurality of proximally directed surfaces in a radial direction relative to the longitudinal axis.

According to another embodiment, the body comprises a plurality of protrusions protruding from a surface of the body in the direction transverse to the longitudinal axis; and each protrusion of the plurality of protrusions comprises one of the plurality of proximally directed surfaces.

Preferably, according to another embodiment, the plurality of protrusions partially overlap with each other in the direction of the longitudinal axis.

Preferably, according to another embodiment, the body comprises a stairway-shaped structure; each stair of the stairway-shaped structure is one of the plurality of proximally directed surfaces.

Preferably, according to another embodiment, the plurality of proximally directed surfaces are overlap with each other in the direction of the longitudinal axis.

Preferably, according to another embodiment, the body comprises ratchet teeth extending along a longitudinal axis between the proximal end and the distal end; each ratchet tooth comprises one of the plurality of proximally directed surfaces.

Preferably, according to another embodiment, the body comprises a flexible arm extending from the body in the direction of the longitudinal axis towards the proximal end of the body; the plurality of proximally directed surfaces are arranged on the flexible arm.

A second aspect of the present disclosure provides a housing extending along a longitudinal axis between a proximal end and a distal end; a guard adapter arranged within the housing, the guard adapter is axially movable relative to the housing and rotationally fixed to the housing; a locking member arranged within the housing; the guard adapter comprises a distally directed surface; the locking member comprises a body extending in an axial direction along the longitudinal axis between a proximal end and a distal end; the body comprises a plurality of proximally directed surfaces sequentially arranged next to each other; and each of the plurality of proximally directed surfaces is at a different position in the axial direction from the rest of the plurality of proximally directed surfaces; the locking member is movable relative to the guard adapter between an unlocked position where the distally directed surface of the guard adapter is not aligned with the plurality of proximally directed surfaces in the direction of the longitudinal axis, and a locked position where at least one of the plurality of proximally directed surfaces is overlap with the distally directed surface of the guard adapter in the direction of the longitudinal axis; and wherein when the locking member is in the locked position, the locking member is axially fixed to the housing.

Preferably, according to another embodiment, the plurality of proximally directed surfaces extend perpendicularly relative to the longitudinal axis.

Preferably, according to another embodiment, each of the plurality of proximally directed surfaces is offset from the rest of the plurality of proximally directed surfaces in a radial direction relative to the longitudinal axis.

Preferably, according to another embodiment, each of the plurality of proximally directed surfaces is overlap with the rest of the plurality of proximally directed surfaces in the direction of the longitudinal axis.

Preferably, according to another embodiment, the sub-assembly comprises a delivery member guard telescopically arranged within the housing, and the guard adapter is a part of the delivery member guard.

Preferably, according to another embodiment, the guard adapter is configured to engage with a delivery member guard of the medicament delivery device.

Preferably, according to another embodiment, the sub-assembly comprises a biasing member extending along the longitudinal axis between a proximal end and a distal end; the guard adapter comprises a second distally directed surface adjacent to the proximal end of the biasing member; and the housing comprises a proximally directed surface connected to the distal end of the biasing member.

Preferably, according to another embodiment, the body of the locking member comprises a flexible arm extending from the body in the direction of the longitudinal axis between a proximal end and a distal end; and the plurality of proximally directed surfaces are a part of the flexible arm, and are arranged between the distal end of the flexible arm and the proximal end of the flexible arm.

Preferably, according to another embodiment, the body of the locking member comprises ratchet teeth; and each ratchet tooth comprises one of the plurality of proximally directed surfaces.

Preferably, according to another embodiment, the ratchet teeth are arranged between the proximal end of the flexible arm and the distal end of the flexible arm.

Preferably, according to another embodiment, the body of the locking member comprises a stairway-shaped structure on a surface of the body; and the plurality of proximally directed surfaces are defined by the stairway-shaped structure, so that each of the plurality of proximally directed surfaces is axially offset from the rest of the plurality of proximally directed surfaces.

Preferably, according to another embodiment, the stairway-shaped structure is arranged on the flexible arm between the proximal end of the flexible arm and the distal end of the flexible arm.

Preferably, according to another embodiment, the body of the locking member comprises a ledge extending in a direction transverse to the longitudinal axis; and the flexible arm extends from the ledge.

According to another embodiment, a cut-out is arranged on the body of the locking member; and the flexible arm extends from an edge of the cut-out.

Preferably, according to another embodiment, the locking member is rotatable relative to the guard adapter around the longitudinal axis between the unlocked position and the locked position.

According to another embodiment, the locking member comprises a proximally directed surface extending in the direction of the longitudinal axis and the direction around the longitudinal axis; and the guard adapter comprises a third distally directed surface configured to move along the proximally directed surface of the locking member thereby rotating the locking member from the unlocked position to the locked position.

According to another embodiment, the proximally directed surface of the locking member extends between a first end and a second end; when the locking member is in the unlocked position, the third distally directed surface of the guard adapter is adjacent to the first end of the proximally directed surface of the locking member; and when the locking member is in the locked position, the third distally directed surface of the guard adapter is adjacent to the second end of the proximally directed surface of the locking member.

Preferably, according to another embodiment, the third distally directed surface is the same as the distally directed surface.

Preferably, according to another embodiment, the body of the locking member comprises a tubular portion extending along the longitudinal axis between a distal end and a proximal end.

Preferably, according to another embodiment, the cut-out is arranged on the tubular portion of the body of the locking member.

Preferably, according to another embodiment, the locking member is axially fixed and rotatable around the longitudinal axis relative to the housing.

Preferably, according to another embodiment, the flexible arm comprises a protrusion extending beyond the plurality of proximally directed surfaces in the direction transverse to the longitudinal axis; a recess/cut-out is arranged on a wall of the housing; and the locking member is axially movable relative to the housing along the longitudinal axis between a first axial position where the protrusion of the flexible arm is adjacent to the wall of the housing and a second position where the protrusion of the flexible arm is overlap with the recess/cut-out on the wall of the housing in the direction transverse to the longitudinal axis.

Preferably, according to another embodiment, the flexible arm is radially movable relative to the longitudinal axis and relative to the guard adapter between a first radial position where the protrusion of the flexible arm is positioned out of the recess/cut-out on the wall of the housing and a second radial position where the protrusion of the flexible arm is positioned within the recess/cut-out on the wall of the housing; and the movement of the locking member between the locked position and the unlocked position is defined by the movement of the flexible arm between the first radial position and the second radial position.

Preferably, according to another embodiment, the body of the locking member comprises a flange extending in the direction transverse to the longitudinal axis; the distal end of the biasing member is adjacent to the flange of the locking member.

Preferably, according to another embodiment, the sub-assembly comprises a slider linearly movable relative to the housing in the direction transverse to the longitudinal axis between a holding position and a released position; the slider comprises a first proximally directed surface; and the locking member comprises a distally directed surface adjacent to the first proximally directed surface of the slider when the slider is in the holding position and disengaged with the first proximally directed surface of the slider when the slider is in the released position.

Preferably, according to another embodiment, the slider comprises a second proximally directed surface extending in the direction of the longitudinal axis and the direction transverse to the longitudinal axis; and the guard adapter comprises a third distally directed surface configured to move along the second proximally directed surface of the slider and thereby linearly move the slider from the holding position to the released position.

According to another embodiment, the third distally directed surface is the same as the distally directed surface.

According to another embodiment, the guard adapter comprises a protrusion extending from a wall of the guard adapter in the direction transverse to the direction of the longitudinal axis; and the distally directed surface is a part of the protrusion.

A third aspect of the present disclosure provides a sub-assembly of a medicament delivery device, the sub-assembly comprising: a housing extending along a longitudinal axis between a proximal end and a distal end; a guard adapter arranged within the housing; a locking member arranged within the housing; the guard adapter is axially movable relative to the housing along the longitudinal axis between a retracted position and an extended position; the locking member comprises plurality of locking surfaces; the guard adapter is blocked by one of the plurality of locking surfaces when the guard adapter moves to the extended position from the retracted position, thereby the guard adapter is blocked from moving from the extended position to the retracted position.

Preferably, according to another embodiment, the locking member is movable between an unlocked position where the plurality of locking surfaces are axially offset to the guard adapter, and a locked position where at least one of the plurality of locking surfaces is axially aligned with the guard adapter.

Preferably, according to another embodiment, the locking member is rotatable around the longitudinal axis between the unlocked position and the locked position by an axial movement of the guard adapter relative to the housing.

Preferably, according to another embodiment, the locking member is radially movable relative to the longitudinal axis between the unlocked position and the locked position.

Preferably, according to another embodiment, the locking member is axially movable relative to the housing between the unlocked position and the locked position.

Preferably, according to another embodiment, the locking member is axially movable relative to the housing between the unlocked position and the locked position by an axial movement of the guard adapter relative to the housing.

Preferably, according to another embodiment, the locking member is releasably fixed to the housing; and the locking member is axially movable relative to the housing between the unlocked position and the locked position by a biasing member.

Preferably, according to another embodiment, an axial movement of the guard adapter relative to the housing is configured to release the locking member from the housing thereby the locking member is biased from the unlocked position to the locked position.

Preferably, according to another embodiment, the locking member comprises a ratchet extending in the longitudinal direction; and the plurality of locking surfaces are surfaces of the ratchet.

Preferably, according to another embodiment, the locking member comprises a stairway-shaped structure; and the plurality of proximally directed surfaces are defined by the stairway-shaped structure, thereby each of plurality of proximally directed surfaces is axially offset to one another.

A fourth aspect of the present disclosure provides a sub-assembly of a medicament delivery device, the sub-assembly comprising the delivery member guard locking member according as mentioned above, and: a housing extending along a longitudinal axis between a proximal end and a distal end; a guard adapter arranged within the housing, wherein the guard adapter is axially movable relative to the housing and rotationally fixed to the housing; the delivery member guard locking member arranged within the housing; the guard adapter comprises a distally directed surface; the locking member is movable relative to the guard adapter between an unlocked position where the distally directed surface of the guard adapter is not aligned with the plurality of proximally directed surfaces in the direction of the longitudinal axis, and a locked position where at least one of the plurality of proximally directed surfaces is overlap with the distally directed surface of the guard adapter in the direction of the longitudinal axis; and when the locking member is in the locked position, the locking member is axially fixed to the housing.

Preferably, according to another embodiment, the medicament delivery device is an injection device, an inhalation device, or a medical sprayer.

Preferably, according to another embodiment, the injection device can be an autoinjector, an on-body injector, a safety syringe or a manual pen-type injector.

Preferably, according to another embodiment, the delivery member can be a needle or a spray nozzle.

Generally, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIG. 1 schematically shows a side view of a housing of the sub-assembly of the present disclosure.

FIG. 2 schematically shows a perspective view of components of the sub-assembly of the present disclosure in one embodiment.

FIG. 3 schematically shows a perspective views of the components of FIG. 2 connected to a delivery member guard of a medicament delivery device.

FIG. 4 schematically shows a perspective view of a locking member of the sub-assembly of the present disclosure in one embodiment.

FIG. 5 schematically shows a perspective view of the locking member of FIG. 4 assembled to a housing part of the sub-assembly of the present disclosure in one embodiment.

FIGS. 6A-C schematically show cross-section views of an operation sequence of the sub-assembly of the present disclosure in one embodiment.

FIG. 7A schematically shows a perspective view of the locking member of FIG. 4 in another embodiment.

FIG. 7B schematically shows a perspective view of a guard adapter of the sub-assembly of the present disclosure that can be used with the locking member of FIG. 7A.

FIG. 8 schematically shows a perspective view of the locking member of FIG. 4 assembled with a slider of the sub-assembly of the present disclosure.

FIG. 9 schematically shows a perspective view of the slider of FIG. 8 and the guard adapter of the sub-assembly of the present disclosure.

FIG. 10 schematically shows a perspective view of components of the sub-assembly of the present disclosure in another embodiment.

FIG. 11 schematically shows a cross-section view of a guard adapter and a housing of the sub-assembly of the present disclosure in another embodiment.

FIG. 12 schematically shows a perspective view of the guard adapter of FIG. 11.

FIGS. 13A-B schematically show perspective views of a locking member of the sub-assembly of the present disclosure in another embodiment.

### DETAILED DESCRIPTION

A first aspect of the present disclosure, illustrated by FIGS. 4-7A and FIGS. 13A-13B, discloses a delivery member guard locking member 3; 13 comprising a body 30; 130 extending in an axial direction along a longitudinal axis L between a proximal end and a distal end. The body 30; 130 comprises a plurality of proximally directed surfaces 35, 35′; 135, 135′ sequentially arranged next to each other; and each of the plurality of proximally directed surfaces 35, 35′; 135, 135′ is at a different position in the axial direction from the rest of the plurality of proximally directed surfaces 35, 35′; 135, 135′. The plurality of proximally directed surfaces 35, 35′; 135, 135′ act as locking surfaces. In a preferred example, the plurality of proximally directed surfaces 35, 35′; 135, 135′ extend perpendicularly relative to the longitudinal axis L. In this example, a movement of the medicament delivery member guard in the distal direction relative to the delivery member guard locking member 3; 13 can be blocked by any one of the plurality of proximally directed surfaces 35, 35′; 135, 135′. In another example, each of the plurality of proximally directed surfaces is formed by a pair of proximally directed surfaces angled to each other, for example, the pair of proximally directed surfaces can formed a “V” shaped valley-like locking surface. In this example, the each of the pair of proximally directed surfaces are not perpendicular relative to the longitudinal axis L.

In one example, each of the plurality of proximally directed surfaces 35; 135 is offset to from the others in a radial direction relative to the longitudinal axis L. For example, the body of the delivery member guard locking member comprises a plurality of protrusions protruding from a surface of the body in a direction transverse to the longitudinal axis L; and each protrusion of the plurality of protrusions comprises one of the plurality of proximally directed surfaces. In a preferred example, the plurality of protrusions partially overlap with each other in the direction of the longitudinal axis L. In another preferred example, the body 30 comprises a stairway-shaped structure 35; 135 wherein each stair of the stairway structure-shaped 35; 135 comprises one of the plurality of proximally directed surfaces 35; 135. In another example, each of the plurality of proximally directed surfaces 35′; 135′ overlaps with the others in the direction of the longitudinal axis L. In another preferred example, the body comprises ratchet teeth 35′ 135′ extending along a longitudinal axis L between the proximal end and the distal end. Each ratchet tooth comprises one of the plurality of proximally directed surfaces 35′ 135′.

The plurality of proximally directed surfaces 35, 35′; 135, 135′ is configured to block a movement of a delivery member guard of a medicament delivery device in the distal direction of the body along the longitudinal axis L after use of the medicament delivery device by one of the plurality of proximally directed surfaces 35, 35′; 135, 135′ that is dependent on a length of the delivery member guard or an end position of the delivery member guard relative to a housing of the medicament delivery device. That is, the delivery member guard locking member 3; 13 of the present disclosure is compatible to be used with medicament delivery devices with different lengths of delivery member guard; or that are arranged with different delivery member guard end positions without significant adjustment of the dimensions and/or structure of the delivery member guard locking member 3; 13.

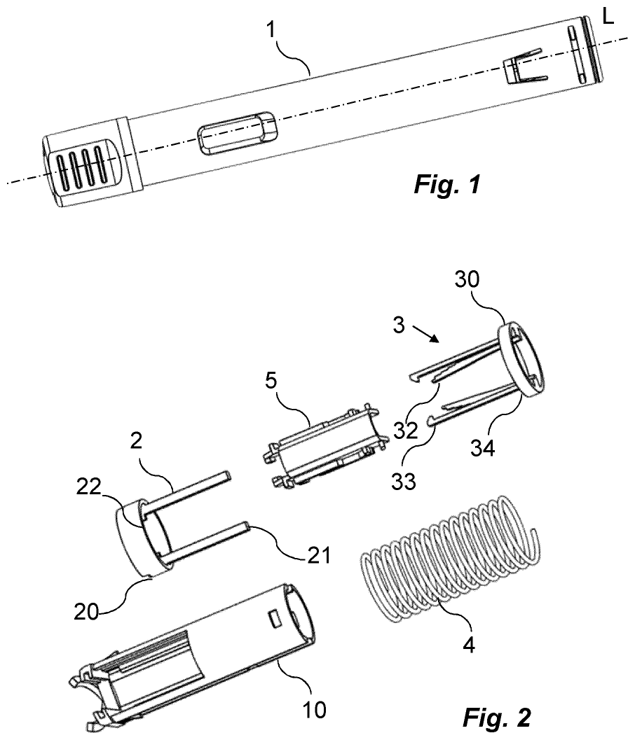

Furthermore, FIGS. 1-13B illustrate a second aspect of the present disclosure, namely a sub-assembly of a medicament delivery device. The sub-assembly comprises the delivery member guard locking member 3; 13 of the first aspect of the present disclosure, which will be named “locking member” hereafter. The sub-assembly comprises a housing 1 extending along the longitudinal axis L between a proximal end and a distal end; a guard adapter 2; 12 arranged within the housing 1; and the locking member 3; 13. When the locking member 3; 13 is attached to the sub-assembly as described below, the longitudinal axis L of the locking member 3; 13 is the same as the longitudinal axis L of the housing 1, or is parallel to the longitudinal axis L of the housing 1.

The housing 1 is configured to accommodate a medicament container of the medicament delivery device, and most of the components that form the medicament delivery device. The housing 1 can be formed in any suitable shape for the medicament delivery device. Preferably, so as to snugly fit the shape of the medicament container, the housing 1 is tubular. The housing 1 can be one single-piece component or can be assembled with a plurality of separate components, depending on the manufacturing method, for example. In one example, the housing 1 comprises an inner housing 10; 110 and an outer shell. The inner housing 10; 110 and the outer shell are attached together in a completely assembled medicament delivery device. On the other hand, when the housing is one single-piece component, the inner housing and the corresponding outer shell would be different parts of that single-piece component.

The guard adapter 2; 12 is axially movable relative to the housing 1 and rotationally fixed to the housing 1, for example, by an engagement between the guard adapter and the housing such as a slot-rib engagement. The guard adapter 2; 12 is configured to connect with a delivery member guard of the medicament delivery device or act as a delivery member guard of the medicament delivery device. A delivery member guard of the medicament delivery device is normally within the housing 1 and telescopic between a retracted position where a delivery member of the medicament delivery device is not covered by the delivery member guard and an extended position where the delivery member of the medicament delivery device is covered by the delivery member guard. The delivery member guard is configured to cover the delivery member of the medicament delivery device when a cap of the medicament delivery device has been removed; therefore, the delivery member guard normally will be biased to the extended position. The biasing force can be provided by a spring 4 or any other suitable biasing element, e.g. an elastic arm.

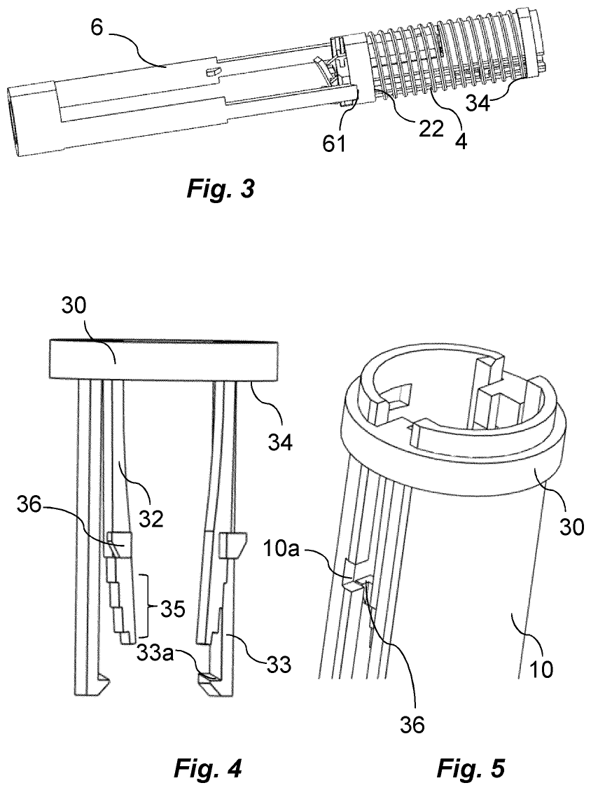

In examples where the guard adapter 2 is configured to connect with a delivery member guard of the medicament delivery device, as shown in FIGS. 2-3, the delivery member guard of the medicament delivery device is not a part of the sub-assembly of the present disclosure. In this example, the guard adapter 2 can be attached to the delivery member guard 6 (preferably at a distal end of the delivery member guard); or as shown in FIG. 3, the distal end 61 of the delivery member guard 6 can be configured to abut a proximally directed surface 20 of the guard adapter 2. In this example, the guard adapter 2 comprises a body comprising a proximally directed surface 20 for engaging with the delivery member guard 6. The guard adapter 2 comprises a distally directed surface 21. The guard adapter 2 optionally comprises a second distally directed surface 22, as shown in FIG. 2, and a third distally directed surface 23, as shown in FIG. 9.

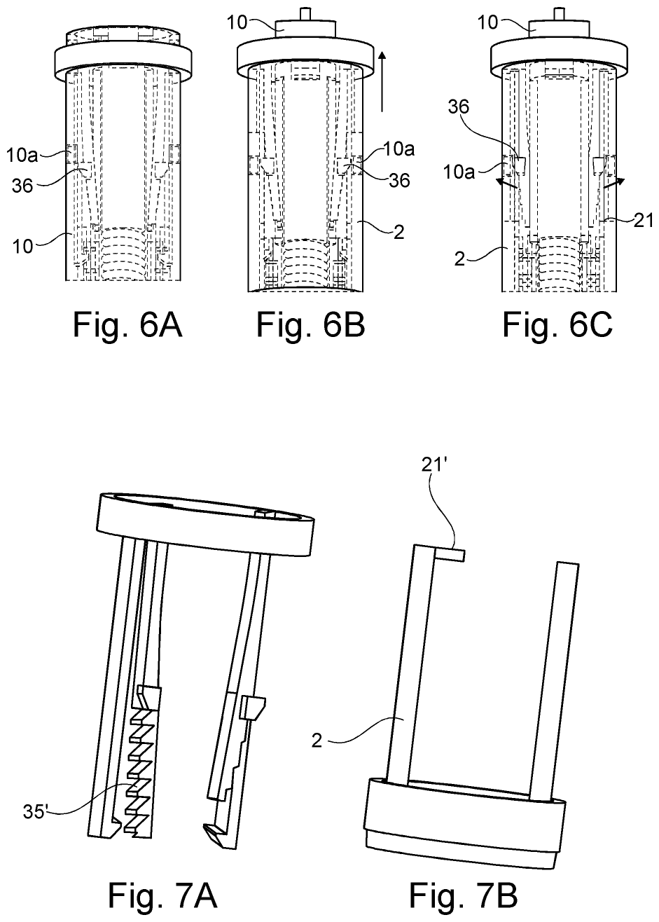

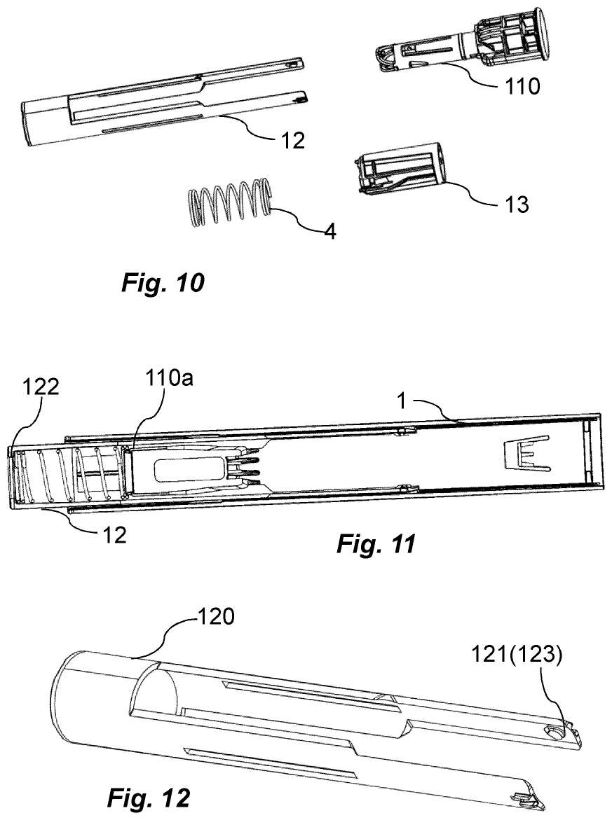

On the other hand, as shown in FIG. 10 and FIG. 12, the guard adapter 12 can be the delivery member guard of the medicament delivery device. Therefore, in this example, the delivery member guard of the medicament delivery device is a part of the sub-assembly of the present disclosure. In the example that the guard adapter 12 acts as the delivery member guard, the guard adapter 12 comprises a body with a tubular portion 120 at the proximal end of the body of the guard adapter 12. The tubular portion 120 is configured to cover the delivery member of the medicament delivery device. The guard adapter 12, similar to the example mentioned above, comprises a distally directed surface 121, and optionally, a second distally directed surface 122. In this presented example, the corresponding third distally directed surface 123 is the same surface of the distally directed surface 121 of the guard adapter 12. The sub-assembly may comprise a biasing member 4 that extends along the longitudinal axis between a proximal end and a distal end. In one example, the proximal end of the biasing member 4 is adjacent to the second distally directed surface 22; 122 of the guard adapter 2; 12; and the distal end of the biasing member is connected to a proximally directed surface of the housing 1. In one example, the distal end of the biasing member is in contact with the proximally directed surface 110a of the housing 1; in another example, the distal end of the biasing member is adjacent to an element that is movable relative to the housing 1. The element is configured to axially move relative to the housing at a certain distance and then engage with the housing 1; the distal end of the biasing member is thus indirectly engaged with the proximally directed surface of the housing 1, as shown in FIG. 3 and FIGS. 6A-6C. Alternatively, the guard adapter is configured to attach to a delivery member guard of the medicament delivery device.

The guard adapter 2, 12 can be formed in any suitable shape dependent on the shape of the delivery member guard of the medicament delivery device and the shape of the housing. For example, the guard adapter 2, 12 may comprise a tubular shaped, or ring-shaped portion with/without one or more distally extending arms.

As mentioned above, in the preferred example, the locking member 3; 13 comprises the body 30; 130. The body 30; 130 comprises the plurality of proximally directed surfaces 35, 35′; 135, 135′ perpendicular to the longitudinal axis L and spaced apart from each other in the direction of the longitudinal axis L and sequentially arranged next to each other. In one example, each of the plurality of proximally directed surfaces 35; 135 is offset relative to the rest of the plurality of proximally directed surfaces in the direction of the longitudinal axis L. In another example, each of the plurality of proximally directed surfaces 35′; 135′ overlaps with the others in the direction of the longitudinal axis L. The locking member 3; 13 is movable relative to the guard adapter 2; 12 between an unlocked position where the distally directed surface 21, 21′; 121 of the guard adapter is not aligned with the plurality of proximally directed surfaces 35, 35′; 135, 135′ in the direction of the longitudinal axis L, and a locked position where at least one of the plurality of proximally directed surfaces 35, 35′; 135, 135′ overlaps with the distally directed surface 21, 21′; 121 of the guard adapter 2; 12 in the direction of the longitudinal axis L. When the locking member 3; 13 is in the locked position, the locking member 3; 13 is axially fixed to the housing 1 (i.e. immovable relative to the housing 1).

The plurality of proximally directed surfaces 35, 35′; 135, 135′ act as locking surfaces. Each of the plurality of proximally directed surfaces 35, 35′; 135, 135′ is configured to selectively block the guard adapter 2; 12 by abutting on the distally directed surface 21, 21′; 121 of the guard adapter 2; 12, when the locking member 3; 13 is in the locked position. Therefore, any further movement of the delivery member guard to the retracted position from the extended position can be prevented. The locking member 3; 13 with the plurality of proximally directed surfaces 35, 35′; 135, 135′ sequentially arranged next to each along the longitudinal axis L can therefore lock the delivery member guard in different axial positions.

For example, different batches of medicament delivery devices or different types of medicament delivery devices may comprise delivery member guards with different lengths; the locking member with the plurality of proximally directed surfaces 35, 35′; 135, 135′ can act as a universal delivery member guard lock being used for different medicament delivery devices with different delivery member guards that have different lengths within a certain range. Manufacturing efficiency can be thus improved. The tolerable range is dependent on the design. Furthermore, medicament delivery devices may comprise an automatic delivery member extending function; for example, if the medicament delivery device is an injection device, a needle as the delivery member can be automatically inserted into an injection site. For those medicament delivery devices with an automatic delivery member extending function, the delivery member guards of those medicament delivery devices normally will extend further in the proximal position after the medicament delivery operation is completed, so that the further extended delivery member (for performing the automatic delivery member extending function) can be covered. The locking member with the plurality of proximally directed surfaces 35, 35′; 135, 135′ can also be used in medicament delivery devices with delivery member guards having the same length and with/without the automatic delivery member extending function.

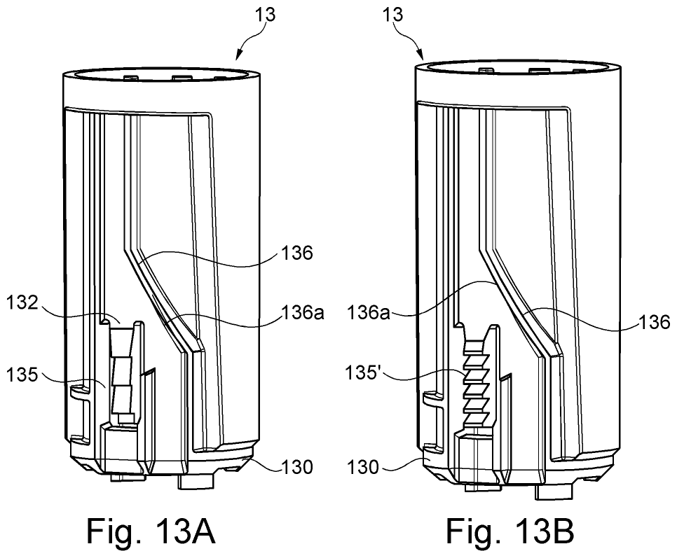

The body 30; 130 of the locking member 3; 13 may comprise ratchet teeth 35′; 135′, and one of the plurality of proximally directed surfaces 35, 35′; 135, 135′ is arranged on each of the ratchet teeth 35′; 135′, as shown in FIG. 7A and FIG. 13B. Alternatively, the body 30; 130 comprises a stairway-shaped structure 35; 135 arranged on a surface of the body 30; 130 in the direction transverse to the longitudinal axis L, as shown in FIG. 4 and FIG. 13A. Furthermore, the ratchet teeth of the body of the locking member 3; 13 can also form a stairway-shaped structure.

In one example, the body 30; 130 optionally comprises a flexible arm 32; 132 extending in from the body 30; 130 in the direction of the longitudinal axis L between a proximal end and a distal end. The plurality of proximally directed surfaces 35, 35′; 135, 135′ are a part of the flexible arm 32; 132 and arranged between the distal end of the flexible arm 32; 132 and the proximal end of the flexible arm 32; 132. In this example, the ratchet teeth 35′; 135′ and/or the stairway-shaped structure 35; 135 is arranged between the proximal end of the flexible arm 32; 132 and the distal end of the flexible arm 32; 132.

The flexible arm 32; 132 is configured to enable the guard adapter 2; 12 to easily move past the plurality of proximally directed surfaces 35, 35′; 135, 135′ in the proximal direction. In this example, the locking member 3; 13 can be designed to move to the locked position from the unlocked position when the delivery member guard moves to the retracted position. An example with a detailed explanation will be presented later. Alternatively, the flexible arm 32 is radially movable relative to the longitudinal axis L and relative to the guard adapter 2 between a first radial position and a second radial position. The movement of the locking member from the locked position to the unlocked position can be defined by the radial movement of the flexible arm 32 from the first radial position to the second radial position. An example with a detailed explanation will be presented later.

It should be noted that the flexible arm, as mentioned above, is not necessary for the locking member that the distally directed surface of the guard adapter is not configured to move past the plurality of proximally directed surfaces, or the distally directed surface of the guard adapter is arranged on a flexible portion of the body of the guard adapter, or the locking member that the movement of the locking member between the locked position and the unlocked position is not defined by the radial movement of the flexible arm from the first radial position to the second radial position. For example, the locking member can be rotated from the unlocked position to the locked position, by the guard adapter when the delivery member guard moves to the extended position from the retracted position. In this example, the plurality of proximally directed surfaces can be rotated then aligned to the distally directed surface of the guard adapter in the direction of the longitudinal axis L when the locking member is rotated from the unlocked position to the locked position.

In one example of the sub-assembly, the locking member 3 is axially movable relative to the housing 1 along the longitudinal axis L. The locking member 3 comprises a proximally extending arm 33 extending from the body 30 of the locking member 3 in the proximal direction, and a flange 34 extending in the direction transverse to the longitudinal axis L. The biasing member 4 in this example is positioned between the guard adapter 2 and the locking member 3. The proximal end of the biasing member 4 is adjacent to the second distally directed surface 22 of the guard adapter 2, and the distal end of the biasing member 4 is adjacent to the flange 34 of the locking member 3. The locking member 3 is thus axially movable relative to the housing 1 under the force of the biasing member 4. In this example, the locking member 3 comprises the flexible arm 32 extending from the body of the locking member, and the plurality of proximally directed surfaces 35, 35′ is arranged between the proximal end of the flexible arm 32 and the distal end of the flexible arm 32.

The flexible arm 32 comprises a protrusion 36 extending beyond the plurality of proximally directed surfaces 35, 35′ in the direction transverse to the longitudinal axis L. In this example, a recess/cut-out is arranged on a wall of the housing 1. In a preferred example, the recess/cut-out 10a is arranged on a wall of the inner housing 10 which is a part of the housing 1, as shown in FIG. 5. The locking member 3 is axially movable relative to the housing 1 along the longitudinal axis L between a first axial position where the protrusion 36 of the flexible arm 32 is adjacent to the wall of the inner housing 10, as shown in FIG. 6A, and a second position where the protrusion 36 of the flexible arm 32 overlaps with the recess/cut-out 10a on the wall of the inner housing 10 in the direction transverse to the longitudinal axis L, as shown in FIGS. 6B-6C. In this example, the movement of the locking member 3 from the locked position to the unlocked position is defined by the radial movement of the flexible arm 32 from the first radial position to the second radial position.

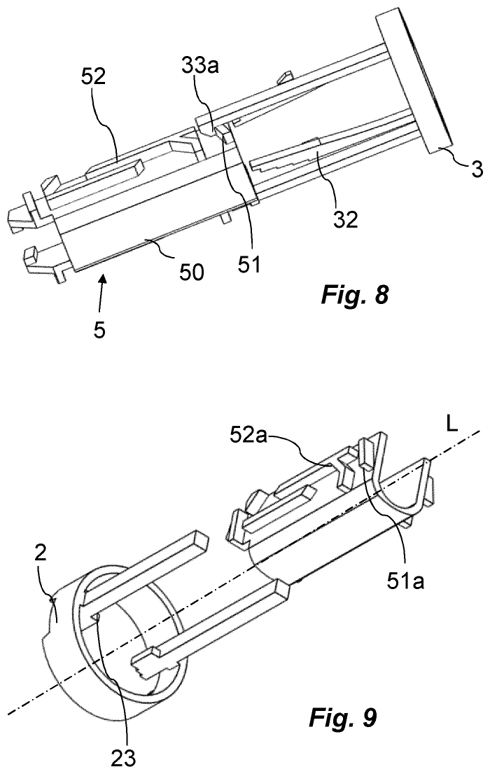

In this example, as shown in FIG. 2, the sub-assembly comprises a slider 5 arranged within the housing 1. The slider 5 is configured to releasably hold the locking member 3. The proximally directed arm 33 of the locking member 3 comprises a distally directed surface 33a. The distally directed surface 33a can be defined by a ledge extending from the proximal end of the proximally directed arm 33 of the locking member 3 in the direction transverse to the longitudinal axis L, or defined by a hook arranged at the distal end of the proximally directed arm 33 of the locking member 3. In this example, the slider 5 is linearly movable relative to the housing 1 in the direction transverse to the longitudinal axis L between a holding position and a released position. The slider 5 comprises a slider body 50, and a first ledge extending from the slider body 50 in the direction transverse to the longitudinal axis L. The first ledge 51 defines a first proximally directed surface 51a of the slider 5. The first proximally directed surface 51a is adjacent to the distally directed surface 33a of the locking member 3 when the slider 5 is in the holding position, as shown in FIGS. 8-9. The slider 5 comprises a second ledge 52 extending from the slider body 50 in the direction transverse to the longitudinal axis L. The second ledge 52 defines a second proximally directed surface 52a extending in the direction of the longitudinal axis L and the direction of the direction transverse to the longitudinal axis L.

The guard adapter 2, in this example, comprises the third distally directed surface 23. In one example, the third distally directed surface 23 is defined by a protrusion protruding from the body of the guard adapter 2, as shown in FIG. 9. The third distally directed surface 23 is configured to move along the second proximally directed surface 52a of the slider 5 when the guard adapter 2 moves together with the delivery member guard 6 in the distal direction related to the housing 1 and the slider 5. The slider 5 is thereby linearly moved from the holding position to the released position where the first proximally direction surface 51a of the slider 5 disengages with the distally directed surface 33a of the locking member 3. The operation sequence of the sub-assembly of this example is shown in FIGS. 6A-6C.

FIG. 6A shows the slider 5 is in the holding position and engaged with the locking member 3 so that the locking member is fixed in the first axial position against the force from the biasing member 4. When the locking member 3 is in the first axial position, the protrusion 36 of the flexible arm 32 is adjacent to the wall of the inner housing 10. When the protrusion 36 is adjacent to the wall of the inner housing 10, the flexible arm 32 is in the first radial position. Therefore, the distally directed surface 35 of the guard adapter 2 is not aligned with the plurality of proximally directed surfaces 35 in the direction of the longitudinal axis L. The axial movement of the guard adapter 2 will thus not be blocked by the plurality of proximally directed surfaces 35 of the locking member 3.

When the delivery member guard moves toward the retracted position, the guard adapter 2 moves relative to the slider 5 (moving direction of the adapter as shown with an arrow on FIG. 6B) and causes the slider 5 to move to the released position, as shown in FIG. 6B. When the slider 5 is in the released position, the locking member 3 disengages from the slider 5 and therefore the locking member 3 is biased towards the distal end of the housing 1 by the biasing member 4 until the locking member 3 abuts the distal end of the housing 1. Once the locking member 3 abuts the distal end of the housing 1 (which also means that the locking member 3 is moved to the second axial position), the locking member 3 is axially fixed to the housing 1. When the locking member 3 is in the second axial position, the protrusion 36 of the flexible arm 32 overlaps with the recess/cut-out 10a on the wall of the inner housing 10 in the direction transverse to the longitudinal axis L.

In a preferred example, when the guard adapter 2 is moved towards the distal end of the housing 1 together with the delivery member guard 6, the body of the guard adapter 2 is positioned between the protrusion 36 of the flexible arm 32 and the recess/cut-out 10a, the protrusion 36 of the flexible arm 32 is thus adjacent to the body of the guard adapter 2, therefore, the flexible arm 32 is kept in the first radial position. When the medicament delivery operation is completed and the medicament delivery device has been lifted from the medicament delivery site, the delivery member guard is moved towards the proximal end of the housing 1 by the force of the biasing member 4 that directly engages with the guard adapter 2. Once the body of the guard adapter 2 moves proximally and is no longer positioned between the protrusion 36 of the flexible arm 32 and the recess/cut-out 10a on the wall of the inner housing 10, the flexible arm 32 is deflected until the protrusion 36 of the flexible arm 32 moves into the recess/cut-out 10a (as shown with arrows in FIG. 6C). The flexible arm 32 is now in the second radial position and at least one of the plurality of proximally directed surfaces 35 overlaps with the distally directed surface 21 of the guard adapter 2 in the direction of the longitudinal axis L, as shown on FIG. 6C. Therefore, further movement of the delivery member guard from the extended position to the retracted position is blocked by the engagement between the distally directed surface 21 of the guard adapter 2 and the at least one of the plurality of proximally directed surfaces 35.

It should be noted that, instead of being defined by the second ledge 52 of the slider 5, the second proximally directed surface 52a of the slider 5 can be defined by an edge of a cut-out on the body 50 of the slider 5.

Furthermore, the slider 5 can be rotatable between the holding position and the released position instead of moving linearly. For example, the first and the second proximally directed surface can be curved surfaces and/or the slider may comprise a round-shaped body.

Furthermore, in this example, the biased movement of the locking member 3 towards the distal end of the housing 1 can be used as an indication mechanism. For example, a sound can be generated when the locking member 3 hits the distal end of the housing 1; or the housing may comprise a window arranged at the distal end of the housing, e.g. the window extends laterally and at least partially around the distal end of the housing or being positioned at the distal end surface that is perpendicular to the longitudinal axis, so that when the locking member 3 abuts the distal end of the housing, a color of the locking member 3 or a mark printed on the locking member 3 can be observed from the window. The indication can be a start indication, e.g. start click; an end click, or a used indication of the medicament delivery device. For example, when the color of the locking member 3 shows up in the window of the housing, that means that the delivery member guard has been lifted (which usually means that the medicament delivery member could be contaminated), so that users can be informed that once a type of color or mark shows in the window, they should not use the medicament delivery device, even if the contained medicament has not yet been expelled.

In another example, the locking member 13 is rotatable relative to the guard adapter 12 around the longitudinal axis between the unlocked position and the locked position. Similar to the examples described above, the locking member 13 in this example comprises a body 130, an optional flexible arm 132, and a proximally directed surface 136a extending in the direction of the longitudinal axis L and the direction around the longitudinal axis L. In a preferred example, the locking member 13 is axially fixed and rotatable relative to the housing 1. For example, the housing 1 may comprise a ledge extending from a surface of the housing 1 in the direction transverse to the longitudinal axis L; and the locking member 13 can be positioned on the ledge, so that an axial movement of the locking member 13 relative to the housing 1 can be prevented by the ledge and the distal end of the housing 1.

The plurality of proximally directed surfaces 135, 135′ are arranged between the distal end of the flexible arm 132 and the proximal end of the flexible arm 132. The proximally directed surface 136a can be defined by a ledge 136 extending from the body 130 of the locking member 13 in the direction transverse to the longitudinal axis L or can be defined by an edge of a cut-out on the body 130 of the locking member 13. The flexible arm 132, in this example, may extend from an edge of a cut-out on the body 130 of the locking member 13, as shown in FIGS. 13A-13B; or may extend from a ledge protruding from the body 130 of the locking member 13 in the direction transverse to the longitudinal axis L.

Similar to the example mentioned above, the guard adapter 12, in this example, comprises a third distally directed surface 123 configured to move along the proximally directed surface 136a of the locking member 13 thereby rotating the locking member 13 from the unlocked position to the locked position. Compared to the above-mentioned example, the guard adapter 12 in this example moves the locking member directly instead of moving the slider to release the locking member. In a preferred example, the locking member 13 is rotated by the guard adapter from the unlocked position to the locked position 12, when the guard adapter 12 moves towards the distal end of the housing 1. Therefore, when the guard adapter 12 moves in the proximal direction when the medicament delivery device has been lifted from the medicament delivery site after the medicament delivery operation is completed, the distally directed surface 121 of the guard adapter 12 moves past the plurality of proximally directed surfaces. Therefore, any further movement of the delivery member guard towards the retracted position can be prevented.

In another example, the body 130 of the locking member 13 comprises a tubular portion, the flexible arm 132 extends from an edge of a cut-out on the body of the locking member 13. The tubular portion of the body 130 extends along the longitudinal axis L between a distal end and a proximal end. The ledge 136 extends on a surface of the tubular portion of the body 130. The tubular portion of the body 130 can provide the benefit that the locking member 13 is easily rotated when a force applied on the proximally directed surface 136a defined by the ledge 136.

In a preferred example, the third distally directed surface 123 is the same as the distally directed surface 121, as shown in FIG. 12.

In the preferred example, the biasing member 4 is positioned between the guard adapter 12 and the housing 1, as shown in FIG. 11 and mentioned above.

It should be noted that the distally directed surface 21, 21′; 121 of the guard adapter 2; 12 can be positioned at the distal end of the body of the guard adapter 2; 12, as shown in FIG. 2; or the body of the guard adapter 2; 12 may comprise a protrusion extending from the body of the guard adapter 2; 12 in the direction transverse to the longitudinal axis L, and thus the distally directed surface 21′; 121 is defined by the protrusion, as shown in FIG. 7B and FIG. 12. These two arrangements of the distally directed surface 21, 21′; 121 of the guard adapter 2; 12 in different examples are both suitable for the locking member with the plurality of proximally directed surfaces that are formed on a stairway-shaped structure or ratchet teeth with stairway-shaped structure. On the other hand, the distally directed surface 21′; 121 that is defined by the protrusion of the guard adapter 2; 12 is suitable for the locking member with the plurality of proximally directed surfaces that are formed on ratchet teeth extending straight along the longitudinal axis L.

The description mainly uses the examples that the plurality of proximally directed surfaces are being perpendicular relative to the longitudinal axis L, or the plurality of proximally directed surfaces extend generally perpendicularly relative to the longitudinal axis L to demonstrate the present disclosure. However, The term “perpendicular” that is used in this application for describing the plurality of proximally directed surfaces is used to emphasize that a mechanical structure that can use any one of the plurality of proximally directed surfaces to keep another component (namely, the guard adapter or the delivery member guard) from moving in the direction of the longitudinal axis L, in at least the proximal direction or the distal direction relative to the housing 1.

Therefore, other embodiments than the ones disclosed above, as long as the plurality of proximally directed surfaces can provide the mechanical structure that can use any one of the plurality of proximally directed surfaces to keep another component (namely, the guard adapter or the delivery member guard) from moving in the direction of the longitudinal axis L, in at least the proximal direction or the distal direction relative to the housing 1, are equally possible within the scope of the inventive concept, as defined by the appended claims.

For example, the blocking function as described above can be provided not only when the plurality of proximally directed surfaces are angled at 90 degrees relative to the longitudinal axis L, and, those surfaces can be, for example, around 70-110 degrees relative to the longitudinal axis L or 80-100 degrees relative to the longitudinal axis, or any other angle that can still keep the counter component immovable in the direction of the longitudinal axis L, in at least the proximal direction or the distal direction relative to the housing 1. The extent to which the angle can be different from exactly perpendicular will depend on factors such as the friction coefficient of the engaged surfaces and the shape of the components.

1-15. (canceled)

16. A sub-assembly of a medicament delivery device, the sub-assembly comprising:
a housing extending along a longitudinal axis between a proximal end and a distal end;
a guard adapter arranged within the housing, wherein the guard adapter is axially movable relative to the housing and rotationally fixed to the housing; and
a delivery member guard locking member arranged within the housing,
wherein the guard adapter comprises a distally directed surface,
wherein the delivery member guard locking member is movable relative to the guard adapter between an unlocked position where the distally directed surface of the guard adapter is not aligned with the plurality of proximally directed surfaces in the direction of the longitudinal axis, and a locked position where at least one of the plurality of proximally directed surfaces is overlap with the distally directed surface of the guard adapter in the direction of the longitudinal axis, and
wherein when the delivery member guard locking member is in the locked position, the delivery member guard locking member is axially fixed to the housing.

17. The sub-assembly according to claim 16, wherein the delivery member guard locking member is rotatable relative to the guard adapter around the longitudinal axis between the unlocked position and the locked position.

18. The sub-assembly according to claim 17, wherein the delivery member guard locking member comprises a proximally directed surface extending in the direction of the longitudinal axis and the direction around the longitudinal axis, and wherein the guard adapter comprises a third distally directed surface configured to move along the proximally directed surface of the locking member thereby rotating the locking member from the unlocked position to the locked position.

19. The sub-assembly according to claim 16, wherein the delivery member guard locking member is axially fixed and rotatable around the longitudinal axis relative to the housing.

20. The sub-assembly according to claim 19, when the delivery member guard locking member comprises a flexible arm, wherein the flexible arm comprises a protrusion extending beyond the plurality of proximally directed surfaces in the direction transverse to the longitudinal axis, a recess/cut-out is arranged on a wall of the housing, and wherein the locking member is axially movable relative to the housing along the longitudinal axis between a first axial position where the protrusion of the flexible arm is adjacent to the wall of the housing and a second position where the protrusion of the flexible arm is overlap with the recess/cut-out on the wall of the housing in the direction transverse to the longitudinal axis.

21. The sub-assembly according to claim 20, wherein the flexible arm is radially movable relative to the longitudinal axis and relative to the guard adapter between a first radial position where the protrusion of the flexible arm is positioned out of the recess/cut-out on the wall of the housing and a second radial position where the protrusion of the flexible arm is positioned within the recess/cut-out on the wall of the housing, and wherein a movement of the locking member between the locked position and the unlocked position is defined by the movement of the flexible arm between the first radial position and the second radial position.

22. The sub-assembly according to claim 16, wherein the plurality of proximally directed surfaces extend generally perpendicularly relative to the longitudinal axis.

23. The sub-assembly according to claim 16, wherein the plurality of proximally directed surfaces are angled at 70-110 degrees relative to the longitudinal axis.

24. The sub-assembly according to claim 16, wherein the plurality of proximally directed surfaces are angled at angled at 80-100 degrees relative to the longitudinal axis.

25. The sub-assembly according to claim 16, wherein each of the plurality of proximally directed surfaces is offset from the rest of the plurality of proximally directed surfaces in a radial direction relative to the longitudinal axis.

26. The sub-assembly according to claim 16, wherein each of the plurality of proximally directed surfaces are overlapped with the rest of the plurality of proximally directed surfaces in the direction of the longitudinal axis.

27. The sub-assembly according to claim 16, wherein the locking member comprises a body, wherein the body of the locking member comprises ratchet teeth, and wherein each ratchet tooth comprises one of the plurality of proximally directed surfaces.

28. The sub-assembly according to claim 16, wherein the ratchet teeth are arranged between the proximal end of the flexible arm and the distal end of the flexible arm.

29. The sub-assembly according to claim 16, wherein the locking member comprises a body, wherein the body of the locking member comprises a stairway-shaped structure on a surface of the body, and wherein the plurality of proximally directed surfaces are defined by the stairway-shaped structure, so that each of the plurality of proximally directed surfaces is axially offset from the rest of the plurality of proximally directed surfaces.

30. The sub-assembly according to claim 29, wherein the stairway-shaped structure is arranged on the flexible arm between the proximal end of the flexible arm and the distal end of the flexible arm.
